(19) United States
(12) Patent Application Publication
(10) Pub. No.: US 2020/0179262 A1
(43) Pub. Date: Jun. 11, 2020

(54) POLYOLEFINS AS FRAGRANCE DELIVERY VEHICLES

(71) Applicants: Dow Global Technologies LLC, Midland, MI (US); Rohm and Haas Company, Collegeville, PA (US)

(72) Inventors: Andrea C. Keenan, Pottstown, PA (US); William A. Cynecki, Midland, MI (US); Chaofang Yue, Dublin, CA (US); Theodore Tysak, Ambler, PA (US); Brian W. Walther, Lake Jackson, TX (US)

(21) Appl. No.: 16/640,209

(22) PCT Filed: Sep. 18, 2018
(86) PCT No.: PCT/US2018/051423

§ 371 Date: Feb. 19, 2020

Related U.S. Application Data
(60) Provisional application No. 62/562,558, filed on Sep. 25, 2017.

(51) Int. Cl. A61K 8/81; A61Q 13/00

(52) U.S. Cl. CPC A61K 8/8111; A61Q 13/00

(57) ABSTRACT

A fragrance release composition comprising: (a) a polyolefin comprising polymerized units of ethylene and at least one C₄-C₁₂ alkene; and (b) a fragrance.

This invention relates to a method for producing a vehicle for delivering fragrances and controlling their release.

Use of polymers as vehicles for fragrance delivery is known. For example, CN102504385A discloses polymer resins used for this purpose, including ethylene-octene copolymer. However, this reference does not disclose the composition described herein.

The problem solved by this invention is the need for improved vehicles for delivery of fragrances.

### STATEMENT OF INVENTION

The present invention provides a fragrance release composition; said composition comprising: (a) a polyolefin comprising 40 to 85 wt % polymerized units of ethylene and 15 to 60 wt % polymerized units at least one C₄-C₁₂ alkene; and (b) a fragrance.

### DETAILED DESCRIPTION

Percentages are weight percentages (wt %) and temperatures are in ° C., unless specified otherwise. Operations were performed at room temperature (20-25° C.), unless specified otherwise. An “alkene” is an unsaturated aliphatic hydrocarbon. Preferably, alkenes have only one double bond. Alkenes may be linear or branched, preferably linear.

A “fragrance” includes any hydrophobic component which provides a pleasant scent. Examples include scents that are floral, ambery, woody, leather, chypre, fougere, musk, vanilla, fruit, and/or citrus. Fragrance oils are obtained by extraction of natural substances or synthetically produced. Fragrances produced may be simple (one essence) or complex (a mélange of essences). Often, the fragrance oils are accompanied by auxiliary materials, such as fixatives, extenders, stabilizers and solvents.

Preferably, the polyolefin comprises polymerized units of ethylene and at least one C₆-C₁₀ alkene, preferably a C₈ alkene, preferably 1-octene. The polyolefin comprises 40 to 85 wt % polymerized units of ethylene and 15 to 60 wt % polymerized units of the alkene; preferably at least 20 wt % alkene, preferably at least 25 wt % alkene, preferably at least 30 wt % alkene; preferably no more than 55 wt % alkene, preferably no more than 50 wt % alkene; preferably at least 45 wt % ethylene, preferably at least 50 wt % ethylene, preferably at least 55 wt % ethylene, preferably at least 60 wt % ethylene, preferably at least 65 wt % ethylene, preferably at least 70 wt % ethylene; preferably no more than 80 wt % ethylene, preferably no more than 75 wt % ethylene, preferably no more than 70 wt % ethylene.

Preferably, the polyolefin has a density (g/cm³) from 0.80 to 0.92, a Melt Index (g/10 min as measured at 2.16 kg @ 190° C.) from 0.3 to 35 and a DSC Melting Peak (° C., Rate 10° C./min) from 30 to 100. Preferably, density is at least 0.85; preferably no more than 0.91, preferably no more than 0.90, preferably no more than 0.89, preferably no more than 0.88. Preferably, Melt Index is at least 0.5, preferably at least 0.7; preferably no more than 30, preferably no more than 15, preferably no more than 10, preferable no more than 5, preferably no more than 3, preferably no more than 2. Preferably, the DSC Melting Peak is no more than 90, preferably no more than 85, preferably no more than 75; preferably at least 35.

Preferably, the weight-average molecular weight (M_{w}) of the polyolefin is from 35,000 to 200,000; preferably at least 40,000, preferably at least 70,000, preferably at least 80,000, preferably at least 90,000, preferably at least 100,000; preferably no more than 170,000, preferably no more than 150,000, preferably no more than 130,000.

The polyolefin may be a random copolymer or a block copolymer. Preferably, the polyolefin is a random copolymer.

Preferably, the fragrance may be delivered to the polyolefin beads using glycol ethers and/or surfactants. Surfactants and glycol ethers may be used to solubilize the fragrance compositions to enhance the delivery to the polyolefin bead. Preferably, glycol ethers useful have the following composition:

R—(OCₙH₂ₙO_{z})OX

wherein R is a substituted or unsubstituted C₁-C₁₂ aliphatic group, a substituted or unsubstituted C₆-C₁₂ aryl group, a group of the formula —C(═O)C₆H₅, or a group of the formula —C(═O)CH₃, n is 2 to 4, z is 1 to 4, and X is —H, —CH₃, —C(═O)CH₃, or —C(═O)C₆H₅. Preferably, R is a substituted or unsubstituted C₁-C₁₀ aliphatic group, preferably an unsubstituted C₂-C₁₀ alkyl group, more specifically an unsubstituted C₂-C₆ alkyl group. In a preferred embodiment, n is 2 to 4, z is 1 to 3, and X is —H. Representative examples of the glycol ether compounds include tripropylene glycol methyl ether, dipropylene glycol n-butyl ether, tripropylene glycol n-butyl ether, dipropylene glycol n-propyl ether, dipropylene glycol phenyl ether, dipropylene glycol methyl ether acetate, propylene glycol n-propyl ether, diethylene glycol monobutyl ether, diethylene glycol n-butyl ether, diethylene glycol monohexyl ether, diethylene glycol hexyl ether, or a combination thereof. Other examples of the second compound may include dipropylene glycol methyl ether, propylene glycol methyl ether, propylene glycol methyl ether acetate, dipropylene glycol methyl ether acetate, or propylene glycol diacetate

The surfactants may be a nonionic, cationic, or anionic material, and it may be a blend of surfactants. Non-limiting examples of surfactants known in the art that may suitably be used include those described in U.S. Pre-Grant publication 2002/0045559. Combinations of surfactants and glycol ethers may be used to enhance the delivery of the fragrance compositions to the polyolefin bead.

Preferably, the amount of fragrance in the fragrance release composition is from 9 to 50 wt % based on the total weight of the composition, preferably at least 10 wt %, preferably at least 12 wt %, preferably at least 15 wt %, preferably at least 20 wt %; preferably no more than 40 wt %, preferably no more than 30 wt %.

### Examples

### Fragrances:

Fragrance 1 (F1): Orange Oil is a product of Sigma Aldrich Corporation, St Louis, Mo., and U.S.A. CAS #8008-57-9
Fragrance 2 (F2): Tropical Breeze is a product of Givaudan Flavor Corporation, East Hanover, N.J., U.S.A.

TABLE 1
Properties Of Polymers

| | | % alkene | | | Melt | DSC | Tg,° C. |
|---|---|---|---|---|---|---|---|
| | | in ethylene- | | Dens., | Index | Melting | DSC |
| | | alkene | | g/cm³ | (dg/ | Peak,° C. | inflec- |
| | | copolymer, | Approx. | ASTM | min, | Rate 10° C./ | tion |
| Material Description | No. | wt % | M_{w} * | D792 | 190C) | min (4) | point (4) |
| Polyolefin Elastomers | P1 | 45 | 115K | 0.857 | 1 | 38 | −58 |
| Ethylene 1-Octene Grades | P2 | 38 | 115K | 0.87 | 1 | 60 | −52 |
| | P7 | 28 | 115K | 0.885 | 1 | 77 | −46 |
| | P3 | 38 | 45K | 0.87 | 30 | 65 | −54 |
| | P8 | 18 | 45K | 0.902 | 30 | 96 | −36 |
| | P9 | 28 | 45K | 0.885 | 30 | 80 | −47 |
| | P10 | 42 | 140K | 0.863 | 0.5 | 47 | −55 |
| ELITE ™ Enhanced | P11 | NA | | 0.964 | 0.85 | 134 | |
| Polyethylene Resins | | | | | | | |
| ATTANE ™ Ultra Low | P12 | NA | | 0.906 | 8.0 | 124 | |
| Density PE Resin | | | | | | | |
| AFFINITY ™ Polyolefin | P4 | 35 | 100K | 0.875 | 3 | 68 | |
| Plastomers and Polyolefin | | | | | | | |
| Elastomers | | | | | | | |
| (ethylene-octene) | P5 | 38 | 10K | 0.87 | 1000 * | 68 | |
| | P13 | 18 | 50K | 0.902 | 7.5 | 98 | |
| | P14 | 12 | 115K | 0.909 | 1 | 106 | |
| | P6 | 38 | 15K | 0.874 | 500 * | 70 | |
| PRIMACOR ™ (20.5% | P15 | NA | 7.5K | 0.958 | 300 | 77 | |
| Acrylic Acid) | | | | | | | |
| (9.7% Acrylic Acid) | P16 | NA | 65K | 0.938 | 10 | 98 | |
| (20% Acrylic Acid) | P17 | NA | 5.5K | 0.955 | 1300 | 75 | |
| DOW LDPE | P18 | NA | 35K | 0.923 | 55 | 110 | |
| AMPLIFY ™ Ethylene-EA | P19 | NA | 110K | 0.932 | 1.3 | 99 | |
| copolymer | | | | | | | |
| Aldrich PE Maleic | P20 | NA | | | | | |
| Anhydride | | | | | | | |
| Aldrich Ethylene-VA | P21 | NA | | | | | |
| ELVAX ™, Ethylene-VA | P22 | NA | 32K | 0.957 | 43 | 63 | |
| (32% VA) 150 | | | | | | | |
| ELVAX ™, Ethylene-VA | P23 | NA | 6K | 0.951 | 400 | 65 | |
| (28% VA) 210W | | | | | | | |
| ELVAX ™, Ethylene-VA | P24 | NA | 15K | 0.951 | 150 | 70 | |
| (28% VA) 220W | | | | | | | |
| ELVAX ™, Ethylene-VA | P25 | NA | 32K | 0.951 | 43 | 74 | |
| (28% VA) 240W | | | | | | | |
| ELVAX ™, Ethylene-VA | P26 | NA | 15K | 0.937 | 150 | 73 | |
| (18% VA) 420 | | | | | | | |
| ELVAX ™, Ethylene-VA | P27 | NA | 52K | 0.941 | 8 | 86 | |
| (18% VA) 450 | | | | | | | |

VA = vinyl acetate;
EA = ethyl acrylate;
PE = polyethylene
* MI estimates based on viscosity
ENGAGE ™ Polyolefin Elastomers; “ELITE ™ Enhanced Polyethylene Resins”; “ATTANE ™ Ultra Low Density Polymers”; “AMPLIFY ™ functional polymers” and AFFINITY ™ Polyolefin Plastomers are products of the Dow Chemical Company, Midland, Michigan, U.S.A.
“Polyolefin Elastomers-ethylene 1-octene grades” where obtained from Aldrich products from Sigma-Aldrich Corporation”.
“PRIMACOR ™ copolymers” are products of SK Global Chemical Co. LTD., Seoul, Korea; “ELVAX ™ copolymer resins are products from DuPont Company, Wilmington, Delaware, U.S.A.
Melt Index measured at (2.16 kg @ 190° C.) ASTM D1238
K = 1,000, i.e., “115K” = 115,000 g/mole

TABLE 2
Bead Integrity In Presence Of Fragrance Oil
Each vial has 1 gram of beads.
Fragrances; 0.1 mls = 100 μl pipetted equivalent to Dosage
wt % of 9.1; 0.5 mls = 500 μl pipetted equivalent
to Dosage wt % 33.3; 1.0 mls = 1000 μl
pipetted equivalent to Dosage wt % 50 in Table 2 below.

| | | | | | | | | Bead |
|---|---|---|---|---|---|---|---|---|
| | | | | | | | | integrity |
| | Performance | F1 | F1 | F1 | F2 | F2 | F2 | after |
| Dosage wt % | Index | 9.1 | 33.3 | 50 | 9.1 | 33.3 | 50 | fragrance |
| P1 | 4.43 | 5 | 5 | 5 | 5 | 4 | 2 | 5 |
| P2 | 4.43 | 5 | 5 | 5 | 5 | 4 | 2 | 5 |
| P7 | 3.57 | 5 | 5 | 4 | 3 | 2 | 1 | 5 |
| P3 | 3.86 | 5 | 5 | 5 | 5 | 3 | 1 | 3 |
| P8 | 3.29 | 5 | 3 | 1 | 5 | 3 | 1 | 5 |
| P9 | 4 | 5 | 5 | 4 | 5 | 3 | 1 | 5 |
| P10 | 3.43 | 5 | 3 | 3 | 5 | 4 | 1 | 3 |
| P11 | 3 | 5 | 3 | 1 | 5 | 1 | 1 | 5 |
| P12 | 3 | 5 | 4 | 2 | 5 | 3 | 1 | 1 |
| P4 | 4 | 5 | 5 | 5 | 5 | 4 | 1 | 3 |
| P5 | 4.14 | 5 | 5 | 4 | 5 | 4 | 3 | 3 |
| P13 | 3.29 | 5 | 3 | 1 | 5 | 3 | 1 | 5 |
| P14 | 2.71 | 5 | 3 | 3 | 4 | 3 | 1 | 0 |
| P6 | 3.14 | 5 | 5 | 5 | 3 | 3 | 1 | 0 |
| P15 | 3.14 | 5 | 4 | 2 | 4 | 3 | 1 | 3 |
| P16 | 2.86 | 5 | 4 | 1 | 4 | 2 | 1 | 3 |
| P17 | 3.43 | 5 | 5 | 2 | 5 | 3 | 1 | 3 |
| P18 | 3 | 5 | 3 | 1 | 3 | 3 | 1 | 5 |
| P19 | 3.14 | 5 | 4 | 2 | 4 | 3 | 1 | 3 |
| P20 | 3 | 5 | 3 | 1 | 5 | 3 | 1 | 3 |
| P21 | 3.86 | 5 | 5 | 5 | 3 | 3 | 3 | 3 |
| P22 | 3.71 | 5 | 4 | 4 | 5 | 4 | 1 | 3 |
| P23 | 3.86 | 5 | 4 | 4 | 5 | 3 | 3 | 3 |
| P24 | 3.86 | 5 | 4 | 4 | 5 | 3 | 1 | 5 |
| P25 | 3.86 | 5 | 4 | 4 | 5 | 3 | 1 | 5 |
| P26 | 3.86 | 5 | 5 | 4 | 4 | 3 | 1 | 5 |
| P27 | 3.71 | 5 | 5 | 4 | 5 | 1 | 1 | 5 |

Performance Rating Assessment

| | Assessment |
|---|---|
| | Rating |
| Fragrance adsorbed | |
| M = most >75% no fluid fragrance observed | 5 |
| P = partial 25-75% slight/minor fragrance fluid | 3 |
| observed | |
| N = none <25% most fragrance fluid observed and not | 1 |
| adsorbed | |
| Integrity | |
| OK = beads appear unchanged in shape or size | 5 |
| SW = Swollen beads | 3 |
| D = Dissolved | 1 |
| A = Agglomerated or sticking together | 0 |

Performance Index is average of Numerical Assessment Ratings for Fragrance Adsorbed and Integrity.
The assessment ratings were based on qualitative determinations by skilled laboratory personnel.
The substrates chosen to measure fragrance release and fragrance longevity were chosen based on Performance Index ratings >4.

TABLE 3
Formulations Of Polyolefin Beads With Fragrance 1

| Composition | Polyolefin | Fragrance |
|---|---|---|
| 1 | 1 gram P1 | 1 gram F1 |
| 2 | 1 gram P2 | 1 gram F1 |
| 3 | 1 gram P3 | 1 gram F1 |
| 4 | 1 gram P4 | 1 gram F1 |
| 5 | 1 gram P5 | 1 gram F1 |
| 6 | 1 gram P6 | 1 gram F1 |

### Methodology for Preparation of Polyolefin Beads for GC/MS and Headspace Analysis.

1 gram of each type of Polyolefin were placed into a 1 ounce vial and weighed. An equivalent weight of fragrance (1 gram), in this case Orange oil (limonene) was added to each vial, so typically the fragrance oil was adsorbed, and the POE beads adsorbed between 46-53% fragrance oil based on weight.
Two vials of each polyolefin was used for the GC/MS and 2 vials were used for the headspace analysis, resulting in 4 vials, each containing 1 gram for every incremental time analysis to be run. The average values in the table were for the 4 vials per time increment, and represent the average amount of fragrance adsorbed per vial and include the standard deviation. So for the initial evaluation there were 4 sets of 1 gram beads/fragrance, at 7 days there were 4 sets of 5 beads/fragrance, at 14 days there were 4 sets of 1 gram beads/fragrance, etc. There were a total of 6 Polyolefin beads examined and were weighed out for: measurements occurring at Initial, 7 days, 14 days, 21 Days, and 28 days.

### Methodology Preparation and Analysis for Fragrance Using GC-MS (Table 4).

A 1% standard mix of the fragrance was prepared in toluene.

The standard mix was diluted in toluene to make the following concentrations: 10,000 and 1000, 500, 100, 10, and 1 ppm.

Each standard were injected into a microvial in a TDU (thermal desorption tube), directly into the TDU.

A calibration curve was made for the fragrance.

Sample size: 5 grams.

Analysis by headspace GC-MS:
33° C. for 0.5 min of heating prior to introduction into GC-MS (to understand VOCs that might contribute to odor.
Column: DB-Wax (30 m×0.25 mm×0.50 μm).
Column: Rtx-5MS (30 m×0.25 mm×0.25 μm). Units are ppm, vol/vol.

TABLE 4
Headspace GC-MS Analysis Average of two runs
Parts per million of fragrance detected in the headspace

| | | T = 7 | T = 14 | T = 21 | T = 28 |
|---|---|---|---|---|---|
| | T = 0 | days | days | days | days |
| Comparative 6 | 1335 | 1581 | 1227.5 | 1049.5 | 521 |
| Example 4 | 1289 | 199.5 | 1200 | 1018 | 692 |
| Comparative 5 | 1411 | 1385 | 1339.5 | 670 | 248.5 |
| Example 1 | 1253.5 | 1194 | 1180 | 882 | 776 |
| Example 3 | 1317 | 1287 | 1175.5 | 972 | 779.5 |
| Example 2 | 1162.5 | 1287 | 1194 | 1022.5 | 787 |

T = Time

Lowest density (least crystalline) and lowest melt peak as well as most elastic Polyolefin P1 bead demonstrated best performance regarding fragrance release.
Examples 1-4 show higher fragrance release at 28 days (parts per million fragrance measured in headspace between 692-779.5 ppm) compared to the Comparative 5 and Comparative 6 (>600 ppm).
Example 1 (polymer P1) showed the best fragrance longevity using Headspace GC and Extraction performed in Hexanes and averaged over 5 beads/per measurement in duplicate.

### Methodology for Evaluation of Fragrance for BULK Analysis (Table 5).

The bulk levels of the fragrance was also tested at T=0 and T=56 Days by HS-GC-MS.
Bulk samples were prepared by dissolving each bead 20-fold in Hexanes.
The headspace and bulk levels of the fragrance was quantitated using a calibration curve of fragrance in Hexanes (1-1000 ppm).
   BULK Data (beads dissolved and extracting fragrance from bead) (Parts per million in substrate), Concentration, wt/wt

TABLE 5

| | | | T = 7 | T = 14 | T = 21 | T = 28 | T = 48 | T = 56 |
|---|---|---|---|---|---|---|---|---|
| Bead type | | T = 0 | Days | Days | Days | Days | Days | Days |
| P6 | Comparative 6 | 55.4 | 50.2 | 33.7 | 23.7 | 17.9 | 1.3 | 0.1 |
| P4 | Example 4 | 56.9 | 46.7 | 34.7 | 25.4 | 13.2 | 6.5 | 0.7 |
| P5 | Comparative 5 | 50.8 | 42.9 | 32.6 | 22.6 | 10.7 | 3.3 | 0.3 |
| P1 | Example 1 | 51.3 | 47.5 | 41.6 | 33.2 | 18.4 | 13.2 | 4.8 |
| P3 | Example 3 | 51.2 | 49.6 | 35.0 | 31.0 | 18.0 | 2.0 | 0.4 |
| P2 | Example 2 | 52.5 | 46.8 | 38.1 | 26.6 | 20.4 | 4.8 | 2.4 |

Example 1, P1 had slower fragrance release comparatively, therefore longer fragrance longevity with bead integrity.
Information in Table 5 demonstrates fragrance retained in beads . . . and then the release from the beads. Example 1 based on P1 allows fragrance (Limonene) to last up to 56 days. Examples 1-4 show higher fragrance retained at 56 days (parts per million fragrance measured in bead) compared to the Comparative 5 and Comparative 6.

1. A composition comprising: (a) a polyolefin comprising 40 to 85 wt % polymerized units of ethylene and 15 to 60 wt % polymerized units of at least one C₄-C₁₂ alkene; and (b) a fragrance.

2. The composition of claim 1 in which the polyolefin comprises 15 to 60 wt % polymerized units of a C₆-C₁₀ alkene.

3. The composition of claim 2 in which the polyolefin comprises 40 to 80 wt % polymerized units of ethylene and 20 to 60 wt % polymerized units of at least one C₆-C₁₀ alkene.

4. The composition of claim 3 in which the polyolefin has M_{w} from 35,000 to 170,000.

5. The composition of claim 4 in which the polyolefin comprises 20 to 60 wt % polymerized units of 1-octene.

6. The composition of claim 1 in which the polyolefin has a melt index from 0.3 to 35.

7. The composition of claim 6 in which the polyolefin has a density from 0.8 to 0.91 g/cm³.

8. The composition of claim 7 in which the polyolefin comprises 40 to 80 wt % polymerized units of ethylene and 20 to 60 wt % polymerized units of at least one C₆-C₁₀ alkene.

9. The composition of claim 8 in which the polyolefin comprises 20 to 60 wt % polymerized units of 1-octene.
